(19) United States
(12) Patent Application Publication
(10) Pub. No.: US 2007/0026424 A1
(43) Pub. Date: Feb. 1, 2007

(54) Gene profiles correlating with histology and prognosis

(76) Inventors: Charles A. Powell, River Vale, NJ (US); Alain C. Borczuk, Roslyn Heights, NY (US)

Correspondence Address: BAKER & BOTTS L.L.P., 30 ROCKEFELLER PLAZA, 44TH FLOOR, NEW YORK, NY, 10112-4498, US

(21) Appl. No.: 11/404,715

(22) Filed: Apr. 14, 2006

Related U.S. Application Data
(60) Provisional application No. 60/671,871, filed on Apr. 15, 2005.

(51) Int. Cl. C12Q 1/68

(52) U.S. Cl. 435/6

(57) ABSTRACT

The present invention related to methods and kits for evaluating the histology and prognosis of lung cancer by measuring expression levels of specific gene markers. It is based, at least in part, on the discovery of 99 genes that were found to be differentially expressed among lung cancer subtypes, 30 genes which correlate with a high risk, and 12 genes which correlate with a low risk, of cancer death within 12 months.

### CROSS-REFERENCE TO RELATED APPLICATION

This application claims priority to U.S. Provisional Application Ser. No. 60/671,871, filed Apr. 15, 2005, which is hereby incorporated by reference in its entirety herein.

### GRANT INFORMATION

The subject matter of this application was developed, at least in part, using funds from National Institutes of Health Grant No. ES00354, so that the United States Government has certain rights herein.

### 1. INTRODUCTION

The present invention relates to methods and kits for evaluating the histology and prognosis of lung cancer by measuring expression levels of specific gene markers. Certain markers that correlate with survival prognoses in cancers other than lung cancer are also identified.

### 2. BACKGROUND OF THE INVENTION

According to the American Cancer Society website (www.cancer.org), there will be about 174,470 new cases of lung cancer in 2006 (92,700 among men and 81,770 among women). Lung cancer is the leading cause of cancer death in the United States (1). Despite innovations in diagnostic testing, surgical technique, and the development of new therapeutic agents, the five-year survival rate has remained ˜13-15% throughout the past three decades. Factors contributing to the low lung cancer survival rate include the small proportion of patients presenting with resectable disease and chemotherapy response rates ranging from 13-42% in patients with advanced stage disease (2, 3). However, even for patients with resected Stage I lung carcinoma, up to 30% will succumb to their disease within five years. Recent research has been directed towards the identification of patients at high risk for death following resection or chemotherapy; these individuals could be candidates for adjuvant therapy or alternative management strategies. Other than clinical stage, there are no established cancer-specific clinical variables or biomarkers that reliably identify individuals at increased risk for death following either surgical resection for early stage non-small-cell carcinomas or chemotherapy and/or radiation therapy for advanced stage carcinomas.

Recent studies indicate that gene expression profiles of resected tumors can provide insights into lung carcinogenesis (4-6) and may predict risk for recurrence and death in early stage lung carcinomas treated by surgical resection (7, 8). These studies suggest that prognostic information provided by molecular profiling of resected lung tumors may be useful in guiding adjuvant therapy or post-resection surveillance strategies. However, since approximately only 20% of lung cancer patients undergo surgical resection with curative intent (9), the applicability of this strategy may be limited. In contrast, biopsy specimens obtained by computed tomography (CT) guided approaches or by fiber-optic bronchoscopy are available from patients with both resectable and unresectable disease (10). Therefore, approaches to examine gene expression profiles from lung cancer biopsies may identify clinically relevant signatures that offer the potential to be widely applicable to the management of lung cancer patients.

### 3. SUMMARY OF THE INVENTION

The present invention relates to methods and kits for evaluating the histology and prognosis of lung cancer by measuring expression levels of specific gene markers. It is based, at least in part, on the discovery of 99 genes that were found to be differentially expressed among lung cancer subtypes, 30 genes which correlate with a high risk, and 12 genes which correlate with a low risk, of cancer death within 12 months.

Accordingly, in one set of embodiments, the present invention provides for a method of evaluating the histology of a lung cancer specimen, and for using disclosed markers to identify lung adenocarcimona, small cell lung cancer, and squamous cell lung cancer. The present invention may be also be used to identify heterogeneous histology in a tissue sample (e.g., squamous cells in an adenocarcinoma tumor), which may be, in non-limiting embodiments, a lung biopsy specimen. The identification of tissue type aids in the selection of appropriate patient treatment.

In additional embodiments, the present invention provides for a method of evaluating the clinical prognosis of a patient suffering from lung cancer, wherein the presence of certain genes are associated with a poorer prognosis and the presence of other genes are associated with a better prognosis. The insight into the probable clinical outcome provided by the present invention assists in making therapeutic choices for a patient. For example, a probable poor prognosis would support decisions for either more aggressive therapy, adjuvant therapy, experimental therapy, or a quality of life decision.

In additional embodiments the present invention provides for the use of gene markers which correlate with prognoses of patients suffering from cancers other than lung cancer.

In still further embodiments, the present invention provides for kits for practicing the methods of the invention. Such kits may contain, for example but not by way of limitation, PCR primers, labeled nucleic acid probes, and/or nucleic-acid bearing chips or blots which may be used to identify one or more genes identified as relevant according to the present invention.

### 4. BRIEF DESCRIPTION OF THE FIGURES

FIG. 1A-B. Scatter plots indicating log gene expression ratios, comparing amplification protocols and comparing biopsy to resected tumor. A. Comparison of targets processed with standard protocol (horizontal axis) and with modified Eberwine protocol (vertical axis). Total RNA was obtained from two microdissected resected tumors and was diluted 1:10 for processing by modified Eberwine procedure. B. Comparison of targets from microdissected resected tumor (horizontal axis) with paired biopsy specimen (vertical axis). The Pearson correlation coefficient for each experiment is indicated in bold, P<0.05 in each instance.

FIG. 2. Kaplan Meier Survival Plots in lung adenocarcinoma patients of representative genes identified in patients undergoing lung biopsy as predictors of cancer death within 12 months. Gene expression data for adenocarcinoma patients were accessed from a dataset that was acquired from 109 patients with early stage resected tumors. For log rank analysis of survival for selected genes, specimens were classified as high expression (n=55) or low expression (n=54) based upon gene expression relative to the median across all specimens; P<0.05 in each instance.

FIG. 3A-F. FHL2 and Cyclin B1 Immunostaining. Two representative biopsy specimens from patient 13 (A-C) and 6 (D-F) were immunostained with antibody to FHL2 (B and E) and Cyclin B1 (C and F). Staining is detectable in tumor cells of specimen 13 but is absent in specimen 6; this correlated with gene signal intensity in these specimens. A and D, H&E stain. Original magnification A-F: ×150.

FIG. 4A-D. Representative biopsy specimens from two patients with non-small-cell carcinoma. A, C: Residual cells from biopsy needles were collected in Dulbecco's Modified Eagle Medium and centrifuged at 2,000 rpm for 5 minutes. A smear was prepared from the pellet, fixed with Fix-Rite 2 (Richard-Allan Scientific, Kalamazoo, Mich.). In A, single tumor cells were seen while in C, clusters of tumor cells were identified. B, D: Core biopsy specimens from the same patients showing morphologically similar tumor cells, indicated by arrows. (A-D, hematoxylin and eosin stain, original magnification 200×).

### 5. DETAILED DESCRIPTION OF THE INVENTION

For clarity, and not by way of limitation, the detailed description of the invention is divided into the following subsections:

(i) genes correlating with histology;

(ii) genes correlating with prognosis;

(iv) methods of evaluating gene expression; and

(v) kits.

5.1 Genes Correlating with Histology

In one set of embodiments, the present invention provides for a method of evaluating the histology of a lung cancer specimen, and for using disclosed markers to identify lung adenocarcinoma, small cell lung cancer, and squamous cell lung cancer.

An increased level of expression of one or more, or preferably at least two, at least three, at least four, at least five, at least six, at least seven, at least eight, at least nine, or at least ten of the following genes: RPS6KA2, BAIAP2, IL1R1, ASL, PRSS8, DAT1, HPN, PHF15, FLJ12443, HLA-DPB1, HOP, LGALS3BP, RUNX1, RBPMS, C11 orf9, HFL1, CEACAM1, RABL4, CAPN2, CLDN4, PON2, MUC1, MICAL2, GPR116, FLJ12443, NpC2, WSB1, CPD, CASP8, STEAP, FOS, TRIM38, ALOX15B (see Table 2, below) correlates positively with presence of lung adenocarcinoma.

Accordingly, the present invention provides for a method for evaluating the histology of a sample comprising lung cells and/or tissue, comprising detecting and/or measuring, in the sample, the expression of one or more, or preferably at least two, at least three, at least four, at least five, at least six, at least seven, at least eight, at least nine, or at least ten of the following genes: RPS6KA2, BAIAP2, IL1R1, ASL, PRSS8, DAT1, HPN, PHF15, FLJ12443, HLA-DPB1, HOP, LGALS3BP, RUNX1, RBPMS, C11 orf9, HFL1, CEACAM1, RABL4, CAPN2, CLDN4, PON2, MUC1, MICAL2, GPR116, FLJI2443, NpC2, WSB1, CPD, CASP8, STEAP, FOS, TRIM38, ALOX15B (see Table 2, below) wherein an increase in the expression of such gene or genes has a positive correlation with the presence of lung adenocarcinoma cells.

An increased level of expression of one or more, or preferably at least two, at least three, at least four, at least five, at least six, at least seven, at least eight, at least nine, or at least ten of the following genes: DKFZp564N1662, SH3GL3, GNAZ, MEIS2, ELOVL2, AF038185, RELN, C11 orf8, AF1Q, KIAA0535, BCL11A, NY-ESO-1, SEPHS1, CDKNIC, BAT8, RIMS2, HEC, FLJ36166, APBA2, TCF3, EYA2, RBP1, L-myc, CDKN2A, SFPQ, KIFC1, ZNF339, CRABP1, RANBP1, STMN1, NCAD, FLJ12377, LMNB1, MGC51028, CENPF, MCM2, INSM1, VRK1, UCHL1, P311, BLM, BCL11A, BCL2, INA, KIAA0186 (see Table 2, below) correlates positively with presence of small cell lung carcinoma.

Accordingly, the present invention provides for a method for evaluating the histology of a sample comprising lung cells and/or tissue, comprising detecting and/or measuring, in the sample, the expression of one or more, or preferably at least two, at least three, at least four, at least five, at least six, at least seven, at least eight, at least nine, or at least ten of the following genes: DKFZp564N1662, SH3GL3, GNAZ, MEIS2, ELOVL2, AF038185, RELN, C11 orf8, AF1Q, KIAA0535, BCL11A, NY-ESO-1, SEPHS1, CDKNIC, BAT8, RIMS2, HEC, FLJ36166, APBA2, TCF3, EYA2, RBP1, L-myc, CDKN2A, SFPQ, KIFC1, ZNF339, CRABP1, RANBP1, STMN1, NCAD, FLJ12377, LMNB1, MGC51028, CENPF, MCM2, INSM1, VRK1, UCHL1, P311, BLM, BCL11A, BCL2, INA, KIAA0186 (see Table 2, below) wherein an increase in the expression of such gene or genes has a positive correlation with the presence of small cell lung carcinoma cells.

An increased level of expression of one or more, or preferably at least two, at least three, at least four, or at least five of the following genes C4.4A, SAP-3, FST, TRIM29, PTPRC (see Table 2, below) correlates positively with presence of squamous cell lung carcinoma.

Accordingly, the present invention provides for a method for evaluating the histology of a sample comprising lung cells and/or tissue, comprising detecting and/or measuring, in the sample, the expression of one or more, or preferably at least two, at least three, at least four or at least five of the following genes: C4.4A, SAP-3, FST, TRIM29, PTPRC (see Table 2, below) wherein an increase in the expression of such gene or genes has a positive correlation with the presence of squamous cell lung carcinoma cells.

In the above methods, when a sample is said to comprise lung cells, it is understood that lung cells are cells found anatomically in the lung or in a tumor which originates or may originate from lung. A population of lung cells may comprise cells of different lineages. In preferred non-limiting embodiments of the invention, the sample is obtained from a lung tumor or metastasis thereof. It is understood that the sample may contain elements such as erythrocytes and white blood cells. In non-limiting embodiments, the percentage of cells histologically identifiable as lung cells or lung cancer cells is more than 50 percent, more than 60 percent, more than 70 percent, more than 80 percent, more than 90 percent, or more than 95 percent.

When the expression of a gene is measured, its level may be compared to a control sample of normal lung tissue, run in parallel, or may be quantified relative to expression of a control gene in the sample (e.g., a “housekeeping” gene such as GAPDH, tubulin, beta actin, etc., as are known in the art), where the relative expression levels in normal cells are ascertained by experiments not run in parallel with the test sample (for example, where control values are predetermined, and, in specific non-limiting embodiments, published or available in a kit).

5.2 Genes Correlating with Prognosis

In additional embodiments, the present invention provides for a method of evaluating the clinical prognosis of a patient suffering from lung cancer, wherein the presence of certain genes are associated with a poorer prognosis and the presence of other genes are associated with a better prognosis.

An increased level of expression of one or more, or preferably at least two, at least three, at least four, at least five, at least six, at least seven, at least eight, at least nine, or at least ten, of the following genes: MYC, TGFB1, SNF1LK, DKK1, LOXL2, OSMR, IRS1, PLOD2, FHL2, BAG2, C14orf78, TRIP-Br2, MTHFD2, SLC7A5, KIF14, OIP5, ADM, KIAA0179, VLDLR, NR4A2, CED-6, CREM, SGCE, CCNB1, NR4A2, FKBP5, ESM1 (and see Table 4, below) correlates positively with a higher risk of shortened survival in a patient suffering from lung cancer (shortened survival means survival for one year or less).

Accordingly, the present invention provides for a method for evaluating the prognosis of a patient suffering from lung cancer, comprising detecting and/or measuring, in a tumor sample from the patient, the expression of one or more, or preferably at least two, at least three, at least four, at least five, at least six, at least seven, at least eight, at least nine, or at least ten of the following genes: MYC, TGFB1, SNF1LK, DKK1, LOXL2, OSMR, IRS1, PLOD2, FHL2, BAG2, C14orf78, TRIP-Br2, MTHFD2, SLC7A5, KIF14, OIP5, ADM, KIAA0179, VLDLR, NR4A2, CED-6, CREM, SGCE, CCNB1, NR4A2, FKBP5, ESM1 (and see Table 4, below), (preferably including one or more of CCNB1, FHL2, LOXL2, IRS1, PLOD2, MTHFD2, TGFB1, and/or TRIP-Br2) wherein an increase in the expression of such gene or genes has a positive correlation with a higher risk of shortened survival.

An increased level of expression of one or more, or preferably at least two, at least three, at least four, at least five, at least six, at least seven, at least eight, at least nine, or at least ten, of the following genes: SCNN1A, GADD45G, SELENBP1, TTF-1, HG3543-HT3739, HLA-DPB1, P8, PLA2G10, HOP, DAT1, RGS16, CTSH (and see Table 4, below) correlates positively with a lower risk of shortened survival in a patient suffering from lung cancer (shortened survival means survival for one year or less, so that there would be a relatively greater likelihood of survival for more than one year).

Accordingly, the present invention provides for a method for evaluating the prognosis of a patient suffering from lung cancer, comprising detecting and/or measuring, in a tumor sample from the patient, the expression of one or more, or preferably at least two, at least three, at least four, at least five, at least six, at least seven, at least eight, at least nine, or at least ten of the following genes: SCNN1A, GADD45G, SELENBP1, TTF-1, HG3543-HT3739, HLA-DPB1, P8, PLA2G10, HOP, DAT1, RGS16, CTSH (and see Table 4, below) (preferably including HLA-DPB1) wherein an increase in the expression of such gene or genes has a positive correlation with a lower risk of shortened survival.

An increased level of expression of one or more, or preferably at least two, at least three, at least four, at least five, at least six, at least seven, at least eight, at least nine, or at least ten, of the following genes: MYC, TGFB1, LOXL2, IRS1, PLOD2, FHL2, TRIP-BR2, MTHFD2, SLC7A5, KIF14, ADM, CCNB1 and ESM1 (and see Table 5, below) correlates positively with a shorter survival relative to a patient having a tumor in which expression of the gene is not increased.

Accordingly, the present invention provides for a method for evaluating the prognosis of a patient suffering from a cancer other than lung cancer, comprising detecting and/or measuring, in a tumor sample from the patient, the expression of at least one, at least two, at least three, at least four, at least five, at least six, at least seven, at least eight, at least nine, or at least ten of the following genes: MYC, TGFB1, LOXL2, IRS1, PLOD2, FHL2, TRIP-BR2, MTHFD2, SLC7A5, KIF14, ADM, CCNB1 and ESM1 (and see Table 5, below), wherein an increase in the expression of such gene or genes has a positive correlation with a higher risk of shorter survival relative to a patient having a tumor in which expression of the gene is not increased. Such patient may be suffering from a cancer other than lung cancer which is, for example, but not limited to, breast cancer, lymphoma, renal cancer, prostate cancer, melanoma, or brain cancer. Alternatively, the patient may be suffering from a cancer other than lung cancer and/or other than breast cancer, other than lymphoma, other than renal cancer, other than prostate cancer, other than melanoma and/or other than brain cancer.

An increased level of expression of one or more, or preferably at least two, at least three, or at least four of the following genes: SCNNIA, HLA-DPB1, DAT1 (LMO3) and CTSH (see Table 5, below) correlates positively with a longer survival relative to a patient having a tumor in which expression of the gene is not increased.

Accordingly, the present invention provides for a method for evaluating the prognosis of a patient suffering from lung cancer, comprising detecting and/or measuring, in a tumor sample from the patient, the expression of one or more, or preferably at least two, at least three, or at least four of the following genes: SCNNIA, HLA-DPB1, DAT1 (LMO3) and CTSH (see Table 5) wherein an increase in the expression of such gene or genes has a positive correlation with a longer survival relative to a patient having a tumor in which expression of the gene is not increased. Such patient may be suffering from a cancer other than lung cancer which is, for example, but not limited to, prostate cancer or ovarian cancer. Alternatively, the patient may be suffering from a cancer other than lung cancer and/or other than prostate cancer and/or other than ovarian cancer.

5.3 Methods of Evaluating Gene Expression

The present invention provides for methods of evaluating (detecting and/or measuring) expression of one or more of the above-mentioned genes in a sample collected from a patient suspected of suffering from or diagnosed with lung cancer.

The sample may be a cell sample or a tissue sample. It may be collected, for example but not by way of limitation, by transthoracic needle biopsy, fiberoptic bronchoscopy, endobronchial biopsy or brushing, or any other technique known in the art. The sample may be a biopsy obtained during conventional surgery or may be a portion of resected tissue. Steps are preferably taken to prevent the degradation of mRNA in the sample; for example, the sample may be maintained at a low temperature (e.g., on ice), rapidly frozen, or rapidly processed.

Gene expression in the sample may be evaluated using standard techniques. Preferably, gene expression may be evaluated by quantitative Polymerase Chain Reaction (“PCR”) using standard laboratory methods. Gene expression may be evaluated, for example but not by way of limitation, using a matrix-assisted laser desorption ionization time-of-flight mass spectrometry, using for example the MassARRAY™ system by SEQUENOM® (www.sequenom.com) (48). Alternatively, gene expression may be evaluated by dot blot, Northern blot, or Western blot analysis, also using standard techniques.

5.4 Kits

In still further embodiments, the present invention provides for kits for practicing the methods of the invention. Such kits may contain, for example but not by way of limitation, PCR primers, labeled nucleic acid probes, and/or nucleic-acid bearing chips or blots which may be used to identify one or more genes identified as relevant according to the present invention.

Said kit may comprise one or more, preferably at least two, at least three, at least four, at least five, at least six, at least seven, at least eight, at least nine, or at least ten, nucleic acid probes and/or sets of PCR primers, or a chip or other matrix material carrying nucleic acid, corresponding to one or more; or preferably at least two, at least three, at least four, at least five, at least six, at least seven, at least eight, at least nine, or at least ten; or up to all of, or less than all of, of the following genes: RPS6KA2, BAIAP2, IL1R1, ASL, PRSS8, DAT1, HPN, PHF15, FLJ12443, HLA-DPB1, HOP, LGALS3BP, RUNX1, RBPMS, C11 orf9, HFL1, CEACAM1, RABL4, CAPN2, CLDN4, PON2, MUC1, MICAL2, GPR116, FLJI2443, NpC2, WSB1, CPD, CASP8, STEAP, FOS, TRIM38, ALOX15B, DKFZp564N1662, SH3GL3, GNAZ, MEIS2, ELOVL2, AF038185, RELN, C11 orf8, AF1Q, KIAA0535, BCL11A, NY-ESO-1, SEPHS1, CDKNIC, BAT8, RIMS2, HEC, FLJ36166, APBA2, TCF3, EYA2, RBP1, L-myc, CDKN2A, SFPQ, KIFC1, ZNF339, CRABP1, RANBP1, STMN1, NCAD, FLJ12377, LMNB1, MGC51028, CENPF, MCM2, INSM1, VRK1, UCHL1, P311, BLM, BCL11A, BCL2, INA, KIAA0186, C4.4A, SAP-3, FST, TRIM29, PTPRC, MYC, TGFB1, SNF1LK, DKK1, LOXL2, OSMR, IRS1, PLOD2, FHL2, BAG2, C14orf78, TRIP-Br2, MTHFD2, SLC7A5, KIF14, OIP5, ADM, KIAA0179, VLDLR, NR4A2, CED-6, CREM, SGCE, CCNB1, NR4A2, FKBP5, ESM1, SCNN1A, GADD45G, SELENBP1, TTF-1, HG3543-HT3739, HLA-DPB1, P8, PLA2G10, HOP, DAT1, RGS16, and/or CTSH (see Tables 2 and 4, below). A nucleic acid “corresponding to” a gene is a nucleic acid that can specifically hybridize to a mRNA transcript of the gene, and for example remains hybridized after stringent washing conditions, such as washing in 0.1×SSC/0.1 percent SDS at 68° C. It need not be the entire gene or the entire cDNA.

In various non-limiting embodiments, the present invention provides for a kit for evaluating a sample comprising lung cells comprising a matrix to which is bound a nucleic acid (preferably a plurality of nucleic acids of the same gene species localized to an area of the matrix in an amount sufficient to generate a detectable signal) corresponding to each of a plurality of genes selected from the group consisting of RPS6KA2, BAIAP2, IL1R1, ASL, PRSS8, DAT1, HPN, PHF15, FLJ12443, HLA-DPB1, HOP, LGALS3BP, RUNX1, RBPMS, C11 orf9, HFL1, CEACAM1, RABL4, CAPN2, CLDN4, PON2, MUC1, MICAL2, GPR116, FLJI2443, NpC2, WSB1, CPD, CASP8, STEAP, FOS, TRIM38, ALOX15B, DKFZp564N1662, SH3GL3, GNAZ, MEIS2, ELOVL2, AF038185, RELN, C11 orf8, AF1Q, KIAA0535, BCL11A, NY-ESO-1, SEPHS1, CDKNIC, BAT8, RIMS2, HEC, FLJ36166, APBA2, TCF3, EYA2, RBP1, L-myc, CDKN2A, SFPQ, KIFC1, ZNF339, CRABP1, RANBP1, STMN1, NCAD, FLJ12377, LMNB1, MGC51028, CENPF, MCM2, INSM1, VRK1, UCHL1, P311, BLM, BCL11A, BCL2, INA, KIAA0186, C4.4A, SAP-3, FST, TRIM29, PTPRC, MYC, TGFB1, SNF1LK, DKK1, LOXL2, OSMR, IRS1, PLOD2, FHL2, BAG2, C14orf78, TRIP-Br2, MTHFD2, SLC7A5, KIF14, OIP5, ADM, KIAA0179, VLDLR, NR4A2, CED-6, CREM, SGCE, CCNB1, NR4A2, FKBP5, ESM1, SCNN1A, GADD45G, SELENBP1, TTF-1, HG3543-HT3739, HLA-DPB1, P8, PLA2G10, HOP, DAT1, RGS16, and CTSH, wherein the number of gene species represented by said plurality of genes preferably constitutes a majority of the total number of gene species bound to the matrix. “Gene species” means a gene having a particular sequence and function; for example, CREM is one gene species amongst the multitude listed above, and GAPDH is a gene species not among the listed “plurality of genes”. As a majority, the plurality of genes may constitute greater than 50 percent, greater than 60 percent, greater than 70 percent, greater than 80 percent, or greater than 90 percent of the total number of gene species represented.

In particular non-limiting embodiment of the invention, a kit may comprise one or more, preferably at least two, at least three, at least four, at least five, at least six, at least seven, at least eight, at least nine, or at least ten, nucleic acid probes, oligonucleotides, and/or pairs of PCR primers, or a chip or other matrix material carrying nucleic acid, corresponding to one or more, preferably at least two, at least three, at least four, at least five, at least six, at least seven, at least eight, at least nine, or at least ten, or all, or less than all, of the following genes: RPS6KA2, BAIAP2, IL1R1, ASL, PRSS8, DAT1, HPN, PHF15, FLJ12443, HLA-DPB1, HOP, LGALS3BP, RUNX1, RBPMS, C11 orf9, HFL1, CEACAM1, RABL4, CAPN2, CLDN4, PON2, MUC1, MICAL2, GPR116, FLJI2443, NpC2, WSB1, CPD, CASP8, STEAP, FOS, TRIM38, and/or ALOX15B, wherein increased expression of these genes is associated with lung adenocarcinoma. In specific non-limiting embodiments, the probes, oligonucletodes, or primers, or the nucleic acids carried on matrix, corresponding to one or a plurality of said genes may be identified as lung adenocarcimona-associated in packaging or instructional material present in the kit, and may, for example, be given an appellation such as a “lung adenocarcinoma panel” or a “lung adenocarcinoma set”, etc.

In other particular non-limiting embodiment of the invention, a kit may comprise one or more, preferably at least two, at least three, at least four, at least five, at least six, at least seven, at least eight, at least nine, or at least ten, nucleic acid probes, oligonucleotides, and/or pairs of PCR primers, or a chip or other matrix material carrying nucleic acid, corresponding to one or more, preferably at least two, at least three, at least four, at least five, at least six, at least seven, at least eight, at least nine, or at least ten, or all, or less than all, of the following genes: DKFZp564N1662, SH3GL3, GNAZ, MEIS2, ELOVL2, AF038185, RELN, C11 orf8, AF1Q, KIAA0535, BCL11A, NY-ESO-1, SEPHS1, CDKNIC, BAT8, RIMS2, HEC, FLJ36166, APBA2, TCF3, EYA2, RBP1, L-myc, CDKN2A, SFPQ, KIFC1, ZNF339, CRABP1, RANBP1, STMN1, NCAD, FLJ12377, LMNB1, MGC51028, CENPF, MCM2, INSM1, VRK1, UCHL1, P311, BLM, BCL11A, BCL2, INA, and/or KIAA0186, wherein increased expression of these genes is associated with small cell lung carcinoma. In specific non-limiting embodiments, the probes, oligonucletodes, or primers, or the nucleic acids carried on matrix, corresponding to one or a plurality of said genes may be identified as small cell lung carcinoma-associated in packaging or instructional material present in the kit, and may, for example, be given an appellation such as a “small cell lung carcinoma panel” or a “small cell lung carcinoma set”, etc.

In other particular non-limiting embodiment of the invention, a kit may comprise one or more, preferably at least two, at least three, at least four, at least five, at least six, at least seven, at least eight, at least nine, or at least ten, nucleic acid probes, oligonucleotides, and/or pairs of PCR primers, or a chip or other matrix material carrying nucleic acid, corresponding to one or more, preferably at least two, at least three, at least four, or at least five, or all, or less than all, of the following genes: C4.4A, SAP-3, FST, TRIM29, and/or PTPRC, wherein increased expression of these genes is associated with squamous cell lung carcinoma. In specific non-limiting embodiments, the probes, oligonucletodes, or primers, or the nucleic acids carried on matrix, corresponding to one or a plurality of said genes may be identified as squamous cell lung carcinoma-associated in packaging or instructional material present in the kit, and may, for example, be given an appellation such as a “squamous cell lung carcinoma panel” or a “squamous cell lung carcinoma set”, etc.

In other particular non-limiting embodiment of the invention, a kit may comprise one or more, preferably at least two, at least three, at least four, at least five, at least six, at least seven, at least eight, at least nine, or at least ten, nucleic acid probes, oligonucleotides, and/or pairs of PCR primers, or a chip or other matrix material carrying nucleic acid, corresponding to one or more, preferably at least two, at least three, at least four, at least five, at least six, at least seven, at least eight, at least nine, or at least ten, or all, or less than all, of the following genes: MYC, TGFB1, SNF1LK, DKK1, LOXL2, OSMR, IRS1, PLOD2, FHL2, BAG2, C14orf78, TRIP-Br2, MTHFD2, SLC7A5, KIF14, OIP5, ADM, KIAA0179, VLDLR, NR4A2, CED-6, CREM, SGCE, CCNB1, NR4A2, FKBP5, and/or ESM1, wherein increased expression of these genes is associated with a higher risk of shortened survival. In specific non-limiting embodiments, the probes, oligonucletodes, or primers, or the nucleic acids carried on matrix, corresponding to one or a plurality of said genes may be identified as shortened survival-associated in packaging or instructional material present in the kit, and may, for example, be given an appellation such as a “shortened survival panel” or a “shortened survival set”, etc.

In other particular non-limiting embodiment of the invention, a kit may comprise one or more, preferably at least two, at least three, at least four, at least five, at least six, at least seven, at least eight, at least nine, or at least ten, nucleic acid probes, oligonucleotides, and/or pairs of PCR primers, or a chip or other matrix material carrying nucleic acid, corresponding to one or more, preferably at least two, at least three, at least four, at least five, at least six, at least seven, at least eight, at least nine, or at least ten, or all, or less than all, of the following genes: SCNN1A, GADD45G, SELENBP1, TTF-1, HG3543-HT3739, HLA-DPB1, P8, PLA2G10, HOP, DATI, RGS16, CTSH, wherein increased expression of these genes is associated with a lower risk of shortened survival. In specific non-limiting embodiments, the probes, oligonucletodes, or primers, or the nucleic acids carried on matrix, corresponding to one or a plurality of said genes may be identified as low risk of shortened survival-associated in packaging or instructional material present in the kit, and may, for example, be given an appellation such as a “longer survival panel” or a “longer survival set”, etc.

In other particular non-limiting embodiment of the invention, a kit may comprise one or more, preferably at least two, at least three, at least four, at least five, at least six, at least seven, at least eight, at least nine, or at least ten, nucleic acid probes, oligonucleotides, and/or pairs of PCR primers, or a chip or other matrix material carrying nucleic acid, corresponding to one or more, preferably at least two, at least three, at least four, at least five, at least six, at least seven, at least eight, at least nine, or at least ten, or all, or less than all, of the following genes: MYC, TGFB1, LOXL2, IRS1, PLOD2, FHL2, TRIP-BR2, MTHFD2, SLC7A5, KIF14, ADM, CCNB1 and ESM1, wherein increased expression of these genes is associated with a shorter survival relative to that of a patient having a tumor in which expression of these genes is not increased. In specific non-limiting embodiments, the probes, oligonucletodes, or primers, or the nucleic acids carried on matrix, corresponding to one or a plurality of said genes may be identified as shortened survival-associated in packaging or instructional material present in the kit, and may, for example, be given an appellation such as a “shorter survival panel” or a “shorter survival set”, etc.

In other particular non-limiting embodiment of the invention, a kit may comprise one or more, preferably at least two, at least three, or at least four, nucleic acid probes, oligonucleotides, and/or pairs of PCR primers, or a chip or other matrix material carrying nucleic acid, corresponding to one or more, preferably at least two, at least three, or at least four, or all, or less than all, of the following genes: SCNNIA, HLA-DPB1, DAT1 (LMO3) and CTSH wherein increased expression of these genes is associated with a lower risk of shortened survival. In specific non-limiting embodiments, the probes, oligonucletodes, or primers, or the nucleic acids carried on matrix, corresponding to one or a plurality of said genes may be identified as low risk of shortened survival-associated in packaging or instructional material present in the kit, and may, for example, be given an appellation such as a “longer survival panel” or a “longer survival set”, etc.

Oligonucleotides to be used as primers or probes specifically bind to their target (corresponding) genes. In non-limiting embodiments, such specific binding may be observed using stringent hybridization conditions, such as e.g., hybridization in 0.5 M NaHPO₄, 7 percent sodium dodecyl sulfate (“SDS”), 1 mM ethylenediamine tetraacetic acid (“EDTA”) at 65° C., and washing in 0.1× SSC/0.1 percent SDS at 68° C. (Ausubel et al., 1989, Current Protocols in Molecular Biology, Vol. I, Green Publishing Associates, Inc., and John Wiley & Sons, Inc. New York, at p. 2.10.3).

### 6. EXAMPLE

### Correlation Between Gene Expression and Clinical

### Features of Lung Cancer

6.1 Methods

Subjects were recruited from a consecutive series of patients referred for transthoracic needle biopsy or bronchoscopy of an undiagnosed lung nodule or mass. Additional inclusion criterion was the diagnosis of a primary lung carcinoma. Tissue specimens were obtained from 26 patients undergoing CT-guided biopsy (n=23, Temno Coaxial Core Biopsy System, Allegiance, McGaw Park, Ill.) or endobronchial brushing (n=3, Cellebrity Endoscopic Cytology Brush, Boston Scientific, Watertown, Mass.) of undiagnosed pulmonary nodules. After needle biopsy and brushing specimens were collected for pathologic diagnosis, the needle or brush containing cells that would otherwise have been discarded was placed into 1 ml RNA extraction buffer (RNeasy Mini kit, Qiagen, Valencia, Calif.). cRNA was generated using the modified Eberwine Protocol

(http://www.affymetrix.com/support/technical/technotes/smallv2_technote.pdf) (15). Compared with the standard amplification protocol, the modified Eberwine procedure incorporates a second cycle of reverse transcription and a second cycle of in vitro transcription.

Biotinylated cRNA was hybridized to the Affymetrix (Santa Clara, Calif.) U95Av2 DNA array, which contains probes for approximately 12,600 human genes. Probe level analysis and normalization to nonmalignant lung tissue was performed using Robust MultiArray Algorithm (16) (Gene Traffic, Iobion, La Jolla, Calif.). Affymetrix Microarray Suite 5.0 was used to determine the designation of present, absent, or marginal for each gene. We excluded from further analysis three arrays of poor quality as demonstrated by fewer than 35% of genes detected as present. Genes were filtered to remove those not present in at least two specimens and genes whose mean log ratio range was less than one. After filtering, 2,194 genes in 23 specimens were used for subsequent analyses. Analyses were performed with BRB-ArrayTools (v. 3.01) (17, 18) and with the Maximum Difference Subset (MDSS) algorithm (http://bioinformatics.upmc.edu/GE2/GEDA.html) (19).

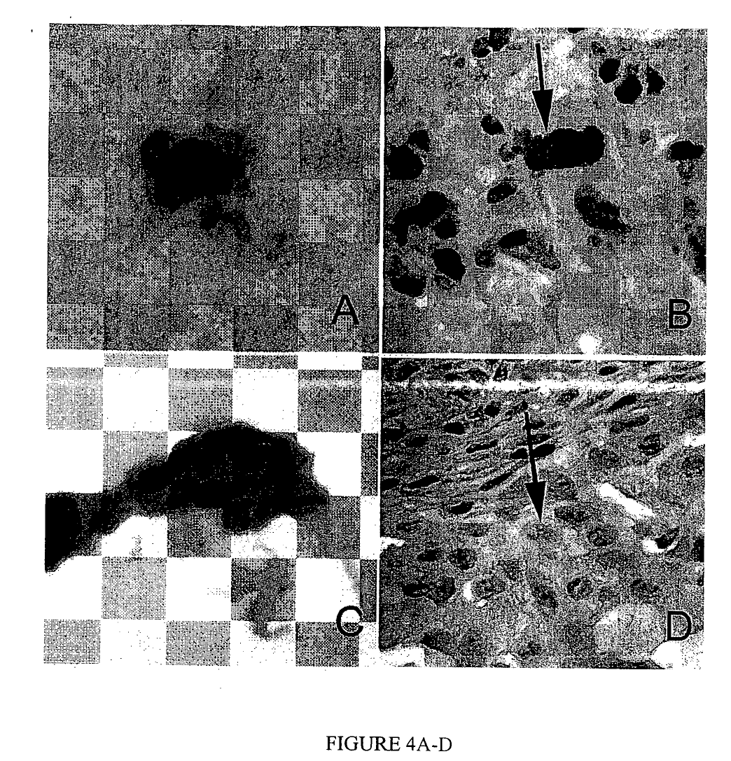

It was not possible to perform cytological analysis on specimens used for gene profiling because the residual specimens for research were immediately placed into lysis buffer. We examined the cellularity of four additional specimens acquired from transthoracic needle biopsy; these were collected using standard procedures but were not processed for gene expression analysis. We determined that 1,000 cells were present in residual specimens obtained from biopsy needles. The morphology of the cells in the residual specimens was similar to the morphology of the tumor cells in paraffin embedded core-biopsy tissues (see FIG. 4A-D). RNA was not specifically quantitated. Based upon cell counts and cRNA yields during processing for expression analysis, we estimate that needle biopsy specimens contained approximately 20-50 ng of total RNA. RNA yields from residual material on bronchoscopy brushings ranged from 500-600 ng.

Biopsy histological diagnosis was acquired from the medical record. Permanent sections were reviewed by a second pathologist, who concurred with the original diagnosis in each instance. The histology was classified using the World Health Organization (WHO) lung tumor classification scheme for small-cell and non-small-cell carcinoma (20). In biopsy and brushing specimens, a diagnosis of adenocarcinoma or squamous cell carcinoma was rendered when there were features associated with differentiation (e.g., gland formation or mucin droplets for adenocarcinoma; keratin or intercellular bridges for squamous carcinoma). If the carcinoma was poorly differentiated, a designation of “non-small-cell carcinoma” was assigned. Clinical information for the subjects was obtained from the medical record and from patients' physicians (Table 1). All procedures were approved by the Columbia University Medical Center Institutional Review Board and informed consent was obtained from participants.

For validation of the histology class prediction model, an independent set of 29 lung carcinoma resection specimens was microdissected and processed for microarray analysis using standard protocols, as reported previously (6). For validation of the outcome class prediction model, gene expression and clinical data from a Massachusetts-based independent cohort of 109 patients with lung adenocarcinoma were accessed from http://www-genome.wi.mit.edu/mpr/lung/. Hu95Av2 CEL files from Massachusetts-based Dataset A (7) were imported into GeneTraffic and processed as above. For the Mantel-Henszel test for survivorship data (log rank test)(21), specimens were classified as high expression or low expression based upon gene expression relative to the median across all specimens. Statistical analyses of survival (22) were performed with SPSS 11.0.

The following datasets were used for analysis: Histology Training Set (n=19 biopsies of adenocarcinoma, squamous, and small-cell carcinoma), Histology Validation Set (n=29 microdissected primary lung carcinoma specimens), Outcome Training Set (n=23 biopsies), Outcome Validation Set (n=109 lung adenocarcinoma patients from Massachusetts-based cohort).

Immunohistochemical staining was performed using antibodies for Cyclin B1 (clone GN5a, Neomarkers, Fremont, Calif.) and FHL2 (Santa Cruz Biotechnology, Santa Cruz, Calif.). Formalin fixed-paraffin embedded biopsy tissue blocks were sectioned at a thickness of 5 μm and dewaxed in xylene and rehydrated through a graded ethanol series and washed with phosphate-buffered saline. For FHL2, antigen retrieval was achieved by heat treatment in a steamer for 40 minutes in 10 mmol/L citrate buffer (pH 6.0); secondary antibody was rabbit anti-goat diluted 1:200 (Vector Labs, Burlingame, Calif.) For Cyclin B1, antigen retrieval was achieved using Protease XXV (Neomarkers, Fremont, Calif.) at 1 mg/ml for 10 minutes at 37° C.; secondary antibody was horse anti-mouse diluted 1:200 (Vector Labs). Before staining the sections, endogenous peroxidase was quenched; for both antibodies, primary antibody incubation was 1 hour at 37° C. (FHL2 1:100, Cyclin B1 1:50).

6.2 Results

Biopsy specimens were adequate for gene expression profiling analysis in 23 of 26 cases. Since our procedures utilized residual material from clinically indicated biopsies, there were no patient complications attributable to the research procedures. A limitation of gene expression profiling of small specimens obtained in this manner is that the number of cells captured does not provide an adequate quantity of total RNA for analysis on Affymetrix oligonucleotide arrays using standard amplification protocols. We therefore instituted the Modified Eberwine procedure, which is an established modification designed to uniformly amplify RNA obtained from small samples for analysis on microarrays.

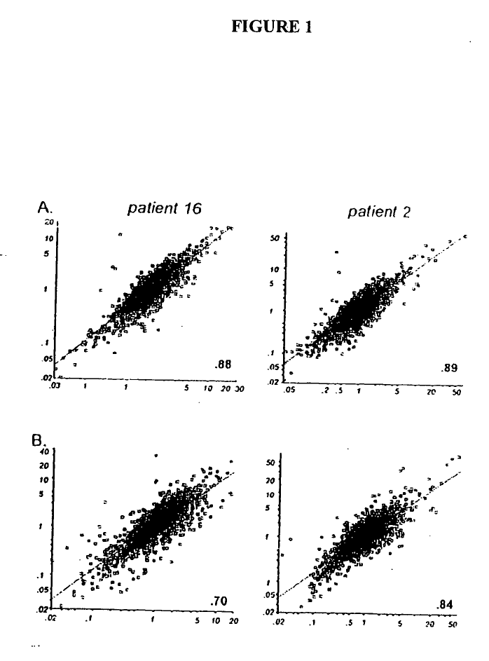

We examined two potential sources of variability in gene profiling of small specimens obtained from diagnostic biopsies—nucleic acid amplification and cellular heterogeneity. To examine the variability introduced by the additional round of amplification in the modified Eberwine procedure, we compared gene expression data of tumor RNA (2 ug) processed with standard procedures with expression of diluted tumor RNA (200 ng) from the same specimen that was processed with the Modified Eberwine protocol. Examination of scatter plots and correlation coefficients show that gene signal intensities were highly similar between the two methods of amplification, as has been shown by other researchers (23-25) (FIG. 1A).

To examine variability introduced by the admixture of cells present in the diagnostic specimens, we compared gene expression data of biopsy material with expression of diluted microdissected tumor RNA from the same patient. The results indicate that the gene expression intensities are similar, but there is more heterogeneity than in the comparison of amplification protocols (FIG. 1B). Since both specimens were processed with the modified Eberwine procedure, the variability was likely attributable to the presence of cellular heterogeneity in biopsy specimens. Compared with microdissected resected tumors that contain >90% tumor cells, the biopsy specimens often contain cells from normal lung, pleura, muscle, skin, inflammatory cells and blood leukocytes in addition to tumor cells. Despite this heterogeneity, we hypothesized that unique tumor specific molecular signatures, (ie. histology classifiers) could be detected in these specimens.

Previous work demonstrates that lung tumor histological subtypes can be distinguished by gene expression profiles (6, 7). To determine if gene expression profiles of lung biopsies could identify specific tumor signatures, we performed Class Comparison using an F-test (26) within BRB-Array Tools to identify 99 genes that were differentially expressed among the histological classes with P<0.01 (Table 2). To address the problem of multiple comparisons in statistical testing, class labels were randomly permuted 1,000 times and a permutation P value <0.01 was associated with each gene in the list. The probability of getting at least 99 genes significant by chance (at the 0.01 level) if there were no real differences between the classes was 0.024. We excluded four lung carcinoma biopsies subtyped as “non-small-cell” from the histology training set cross-validation analysis. The designation of “non-small-cell” encompasses multiple histological subtypes and is not a WHO category for histological classification of resected tumors.

Among the lung histology classifier genes detected in the biopsy specimens, several have been identified in other studies that used the U95A microarray platform. These marker genes include ERBB2, TTF-1, MUC1, BENE, SELENBP1, TGFBR2 (adenocarcinoma); KIF5C, TMSNB, TUBB, FOXG1B, ESPL1, TRIM28 (small-cell carcinoma); and KRT17, KRT6E, BPAG1 (squamous cell carcinoma) (6, 7, 27). To further examine the association of the classifiers with lung cancer histology, we performed Class Prediction testing with a k-nearest neighbor (28) leave-one-out cross-validation. In this procedure, one sample is removed from the training set, a new gene set is generated, from which a classifier is generated, and this classifier is applied to the sample left out. This procedure is repeated for all of the samples. 3-nearest neighbor classifiers generated in this manner correctly predicted the histological class for 13 (68%) of 19 samples. A permutation analysis of the predictor was performed. Based on 1,000 random permutations, the classifier had a P value of 0.035 indicating that the misclassification rate of the predictor was significantly smaller than the misclassification rate of the permutations.

We tested the accuracy of the biopsy histology classifier model by using it to predict the histology of 29 independently obtained lung carcinoma resection specimens (histology validation set). The distribution of the histology validation set was adenocarcinoma (n=22); small-cell (n=2); and squamous cell carcinoma (n=5). The 99 gene histology classifier model was able to accurately predict histology in 25 (86%) of 29 tumors (Table 3). Four of the adenocarcinoma tumors were incorrectly classified as squamous cell carcinomas. Interestingly, histological sections of these tumors showed areas of squamous differentiation within a predominantly glandular tumor and in a previous study, three of these adenocarcinomas segregated with squamous cell carcinomas in an unsupervised clustering procedure (6). Therefore, histological heterogeneity may have accounted for misclassification by histology classifier genes in these tumors. The results of histology training and validation set class prediction analyses indicate that gene expression profiles of lung biopsies were representative of histologically specific subtypes of lung carcinoma.

We examined whether biopsy gene expression signatures could predict another clinically relevant endpoint, prognosis. Of the 23 patients who underwent lung biopsy, six cancer deaths occurred within 12 months. These patients were classified as high risk for early cancer death. We identified genes associated with high risk and low risk outcome using the Maximum Difference Subset (MDSS) algorithm. This tool combines standard statistical tests (pooled variance t-test) and machine prediction learning to identify class predictors with higher specificity and accuracy compared with other classification algorithms (19). In the biopsy dataset, MDSS identified 42 genes associated with cancer death within 12 months (Table 4). We tested the accuracy of these predictors to classify risk for cancer death. The overall outcome training set class prediction accuracy rate was 87% (20 of 23 predicted correctly), with a P value of 0.008 based upon 1,000 random permutations of the class labels.

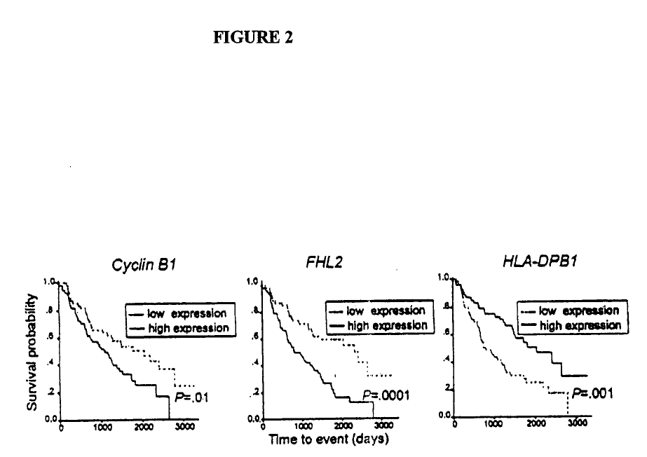

To determine if the outcome classifiers identified in expression profiling of lung cancer biopsies were applicable to other lung cancer gene expression datasets, we examined whether our genes were associated with cancer-free survival in an independent set of homogenized tumors resected from a large cohort of Massachusetts-based lung adenocarcinoma patients (outcome validation set) (7). We determined that 9 of the 42 genes associated with risk for one year cancer death in our outcome training set were associated (positively or negatively) with cancer-free survival in the Massachusetts-based outcome validation dataset, using the log rank test, P<0.05 (FIG. 2). These genes were: CCNB1, FHL2, HLA-DPB1, LOXL2, IRS1, PLOD2, MTHFD2, TGFB1, and TRIPBR2. This result suggests that despite differences in histologic subtypes, specimen types and amplification protocols, selected outcome genes may be applicable to the prediction of lung carcinoma outcome in other patients.

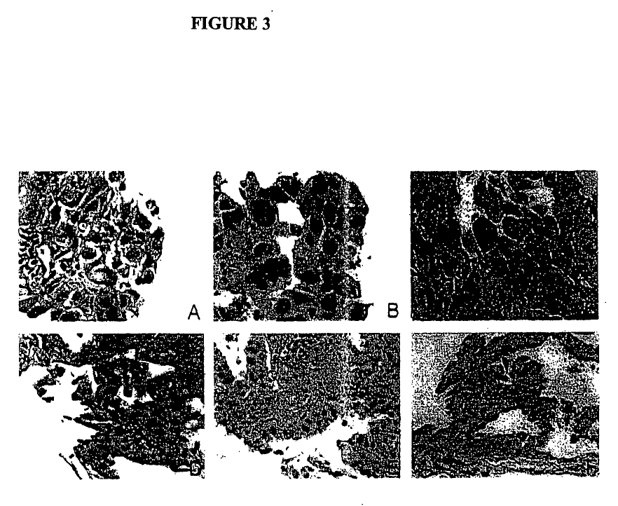

Since tumor behavior may be modulated by signals from the tumor and its surrounding microenvironment, we examined immunolocalization of representative outcome marker proteins to determine if expression was detectable in tumor cells. Antibodies were selected on the basis of commercial availability. Immunoreactivity for both FHL2 (nuclear) and Cyclin B1 (cytoplasmic) was detectable in tumor cells, suggesting that biopsy gene expression signatures are derived from tumor cells (FIG. 3).

6.3 Discussion

Lung cancer biopsy gene expression profiles identify unique tumoral signatures that provide information about tissue morphology and clinical outcome. Using validated methods of gene identification that account for the statistical problems associated with multiple comparisons, the present study identified 42 genes associated with high risk for cancer death within one year. The use of specimens acquired from lung biopsy procedures to identify genes associated with clinical outcome suggests several applications as biomarkers of prognosis or treatment response.

The relevance of the outcome marker genes identified in the biopsy specimens is supported by other studies indicating that several genes are associated with prognosis in patients with lung carcinoma or other carcinomas. Examples include MYC, encoding the nuclear transcription factor c-myc, which functions in cell growth and proliferation and is frequently amplified in lung carcinoma (29). Increased expression of MYC is associated with adverse prognosis in lymphoma and node-negative breast carcinoma (30, 31). CCNB1 encodes the cell cycle regulatory protein Cyclin B1, which regulates the G2/M transition. Increased expression of Cyclin B1 is associated with poor survival in esophageal carcinoma and in non-small-cell lung carcinoma (32, 33). FHL2 encodes four and a half of LIM-only protein, which is a β-catenin binding protein with trans-activation activity (34). FHL2 expression is increased in hepatoblastoma and is associated with Cyclin D1 promoter activation in a β-catenin dependent fashion. While FHL2 is not directly associated with cancer outcome, Cyclin D1 expression is associated with decreased survival in resected lung carcinomas (35). HLA-DPB1, which encodes a human MHC Class II lymphocyte antigen beta chain, was associated with improved survival in our dataset. A similar association was recently reported in a gene profiling study of diffuse large B cell lymphoma specimens. Lower expression of HLA-DPB1 and other MHC class II genes was associated with poor patient survival and decreased tumor immunosurveillance (36).

The five-year survival rate for lung cancer is approximately 15%, which is markedly lower than the rates for other common cancers of the breast, colon and prostate (37). This discrepancy may be due to biological differences such as histological heterogeneity or to the absence of proven screening programs that effectively detect cancers at an early, curable stage. However, even for surgically resected early Stage I non-small-cell lung carcinomas, the recurrence rate is 3-5% annually and the five-year survival rate is approximately 70%. Recent studies suggest that gene expression profiles of early stage lung adenocarcinomas may predict risk for death (7, 8) and therefore may be useful to identify individuals who would be most likely to benefit from systemic therapy delivered before or after resection. Data from early stage lung cancer systemic therapy trials indicate that neoadjuvant chemotherapy combined with radiation therapy (38) and adjuvant chemotherapy (39) may provide a survival benefit for a small proportion of patients. The potential role of lung biopsy gene expression profiling in the management of early stage non-small-cell carcinoma would be to identify patients with high risk tumors who would be most likely to benefit from neoadjuvant systemic therapy. The potential utility of this approach has been demonstrated in breast carcinoma. Gene profiles obtained from breast tumors have been shown to predict a short-term clinical response to neoadjuvant docetaxel (40).

Another potential role for gene profiling of lung cancer biopsies that might be applicable to the large proportion of lung cancer patients with unresectable tumors is selection of chemotherapy agents. Advanced stage non-small-cell carcinomas and small-cell carcinomas are treated with systemic chemotherapy. For non-small-cell lung carcinomas, the average response rate in previously untreated patients ranges widely from 13-42% (2); yet there are no reliable biomarkers to guide the selection of particular regimens to patients who are most likely to benefit. Recent in vitro studies show that the response of lung cancer cells and other cancer cells to single chemotherapy agents can be predicted by distinct gene expression profiles (41, 42). These results suggest that gene profiling may complement decisions regarding the selection of systemic chemotherapeutic agents. This hypothesis is supported by recent B cell lymphoma clinical trials that identified tumor gene expression predictors of patient survival after chemotherapy treatment (43, 44). Interestingly, adverse prognosis genes were associated with a proliferation functional class while favorable outcome was associated with MHC Class II function (43). In our lung biopsy dataset, proliferation genes (CCNB1, MYC, FHL2, NR4A2) and MHC Class II genes (HLA-DPB1) were similarly associated with adverse and favorable outcomes, respectively. Further characterization of the function of these genes in lung carcinogenesis may lead to the development of novel targeted therapies.

Some methodological limitations apply to our approach. First, our use of residual biopsy specimens did not consistently provide enough cellular material for gene expression analysis using standard amplification protocols. Rather, we used a modified protocol that incorporated a second round of amplification and therefore increased the opportunity for variability and inconsistency in the data. However, our validation experiments and those performed by others indicate that experimental variability attributable to amplification procedures is small and that data produced from small specimens are reliable. Our technical adequacy rate was higher than those reported by other studies that examined gene expression profiles of lung and breast biopsies (25, 45). Second, the sample size was relatively small, which may introduce bias and reduce the ability to generalize our results to other lung cancer populations. To address this issue, we examined the ability of the outcome classifier model to predict cancer-free survival in a large independent gene expression dataset of lung adenocarcinoma tumors. Despite differences in tumor specimen composition and in experimental protocols, several of our cancer outcome classifier genes were similarly associated with cancer-free survival in Massachusetts-based lung adenocarcinoma cases. Future prospective validation of the gene classifier model in an independent cohort of patients undergoing biopsy will reduce confounding by technical and clinical factors and will confirm the generalizability of the results. Third, since our dataset was comprised entirely of lung carcinoma biopsies, we could not examine the utility of biopsy gene profiles to distinguish malignant tumors from benign nodules. Recent experience with screening chest CT indicates a high prevalence of nodules (25-66%) of which only a small fraction (1-3%) are malignant (46). While nodule size and interval change in size are useful tools to distinguish malignant from benign lesions, it is possible that gene expression profiles of CT-detected nodules may enhance diagnostic algorithms and the clinical utility of the procedure.

Other reports support the potential utility of biopsy gene profiles in the clinical management of breast carcinoma. Compared with breast biopsies, lung biopsy is associated with a higher risk of complications such as bleeding and pneumothorax. We addressed this risk in our study procedures by utilizing residual specimens from clinically indicated diagnostic lung biopsies; thus no medical risk was attributable to procedures utilized for gene expression analysis of lung biopsies. The gene expression signatures generated by the lung biopsies are robust, clinically relevant, and have the potential to improve lung cancer treatment and outcome. The procedures are safe and feasible; we suggest that the efficacy and utility of this strategy are now appropriate for assessment by prospective clinical trials.

TABLE 1
PATIENT CHARACTERISTICS

| | | | | | Tumor | | | Follow- |
|---|---|---|---|---|---|---|---|---|
| | Age | | | | Size | | Cancer | Up |
| Sample | (yr) | Sex | Pathology | Source | (cm) | Stage | Death | (d) |
| 1 | 62 | M | Adenocarcinoma | ttn | 5.1 | IV | No | 432 |
| 2* | 88 | M | Adenocarcinoma | ttn | 4 | IB | No | 502 |
| 3 | 63 | M | Adenocarcinoma | ttn | 2.6 | IIIA | No | 379 |
| 4 | 67 | F | Adenocarcinoma | ttn | 4.3 | IV | No | 389 |
| 5 | 80 | F | Adenocarcinoma | ttn | 2.5 | IB | No | 108 |
| 6 | 70 | F | Adenocarcinoma | ttn | 2.5 | IV | No | 230 |
| 7 | 61 | F | Squamous | Brush | 2.9 | IA | No | 248 |
| 8 | 77 | F | Squamous | ttn | 2.4 | IIIA | No | 341 |
| 9 | 56 | M | Squamous | ttn | 9.3 | IIIA | No | 59 |
| 10 | 56 | M | Squamous | ttn | 6.7 | IIIA | No | 281 |
| 11 | 69 | M | Squamous | ttn | 4.5 | IIa | No | 328 |
| 12 | 55 | F | Non-small cell | ttn | 10.5 | IIB | Yes | 102 |
| 13 | 66 | M | Squamous | Brush | 4.5 | IIIA | Yes | 259 |
| 14 | 65 | F | Adenocarcinoma | ttn | 1.2 | IIIA | No | 437 |
| 15 | 89 | M | Non-small cell | ttn | 10 | IV | Yes | 54 |
| 16* | 77 | M | Adenocarcinoma | ttn | 2.6 | IB | No | 355 |
| 17 | 85 | F | Adenocarcinoma | ttn | 3.8 | IV | Yes | 442 |
| 18 | 72 | M | Squamous | ttn | 5.2 | IIA | Yes | 58 |
| 19 | 64 | M | Non-small cell | ttn | 4.8 | IV | Yes | 265 |
| 20 | 40 | F | Non-small cell | Brush | 2.5 | IIIB | No | 270 |
| 21 | 55 | M | Adenocarcinoma | ttn | 8.1 | IV | No | 275 |
| 22 | 74 | M | Small cell | ttn | 8 | E | No | 400 |
| 23 | 72 | F | Small cell | ttn | 3.7 | E | Yes | 346 |

Definition of abbreviations:
brush = bronchoscopy brushing;
E = extensive stage;
ttn = transthoracic needle biopsy.
*Resected tumor available for gene expression analysis.

TABLE 2
HISTOLOGY CLASSIFIERS
OF BIOPSY SPECIMENS IDENTIFIED BY F TEST

| Adenocarcinoma | | Small Cell | |
|---|---|---|---|
| Affymetrix ID | Symbol | Affymetrix ID | Symbol |
| 33325_at | RPS6KA2 | 36701_at | DKFZp564N1662 |
| 37760_at | BAIAP2 | 37580_at | SH3GL3 |
| 33218_at | ERBB2 | 35778_at | KIF5C |
| 33754_at | TTF-1 | 38279_at | GNAZ |
| 927_s_at | MUC1 | 41388_at | MEIS2 |
| 1368_at | IL1R1 | 39642_at | ELOVL2 |
| 36528_at | ASL | 36815_at | AF038185 |
| 634_at | PRSS8 | 37530_s_at | RELN |
| 38028_at | DAT1 | 36491_at | TMSNB |
| 37639_at | HPN | 36029_at | C11orf8 |
| 38342_at | PHF15 | 36941_at | AF1Q |
| 33331_at | BENE | 38146_at | KIAA0535 |
| 37405_at | SELENBP1 | 41356_at | BCL11A |
| 41177_at | FLJ12443 | 33637_g_at | NY-ESO-1 |
| 38095_i_at | HLA-DPB1 | 39387_at | SEPHS1 |
| 39698_at | HOP | 39605_att | FOXGIB |
| 37754_at | LGALS3BP | 1787_at | CDKNIC |
| 943_at | RUNXI | 36200_at | BAT8 |
| 38047_at | RBPMS | 38163_at | RIMS2 |
| 33327_at | C11orf9 | 40041_at | HEC |
| 32249_at | HFL1 | 34417_at | FLJ36166 |
| 988_at | CEACAM1 | 39590_at | APBA2 |
| 36076_g_at | RABL4 | 1373_at | TCF3 |
| 37001_at | CAPN2 | 35226_at | EYA2 |
| 35276_at | CLDN4 | 39332_at | TUBB |
| 40504_at | PON2 | 38634_at | RBP1 |
| 38783_at | MUC1 | 1490_at | L-myc |
| 40848_g_at | MICAL2 | 1713_s_at | CDKN2A |
| 34235_at | GPR116 | 41199_s_at | SFPQ |
| 41176_at | FLJ12443 | 38933_at | KIFC1 |
| 39345_at | NpC2 | 36761_at | ZNF339 |
| 40928_at | WSB1 | 38158_at | ESPL1 |
| 34876_at | CPD | 33425_at | TRIM28 |
| 33774_at | CASP8 | 543_g_at | CRABP1 |
| 40297_at | STEAP | 41342_at | RANBP1 |
| 1815_g_at | TGFBR2 | 1782_s_at | STMN1 |
| 1915_s_at | FOS | 2054_g_at | NCAD |
| 35341_at | TRIM38 | 39324_at | FLJ12377 |
| 37430_at | ALOX15B | 37985_at | LMNB1 |
| | | 41084_at | MGC51082 |
| | | 37302_at | CENPF |
| | | 35312_at | MCM2 |
| | | 33157_at | INSM1 |
| | | 39980_at | VRK1 |
| | | 36990_at | UCHL1 |
| | | 39710_at | P311 |
| | | 1544_at | BLM |
| | | 41355_at | BCL11A |
| | | 1909_at | BCL2 |
| | | 37210_at | INA |
| | | 39677_at | KIAA00186 |

Squamous Cell

| Affymetrix ID | Symbol |
|---|---|
| 34301_r_at | KRT17 |
| 41641_at | C4.4A |
| 39016_rat | KRT6E |
| 39015_f_at | KRT6E |
| 40304_at | BPAG1 |
| 35820_at | SAP-3 |
| 38356_at | FST |
| 1898_at | TRIM29 |
| 40518_at | PTPRC |

TABLE 3
PREDICTION OF RESECTED TUMOR HISTOLOGY

| Specimen | Histology | Prediction |
|---|---|---|
| AD20009 | AD | SQ |
| AD20014 | AD | AD |
| AD20033 | AD | AD |
| AD21001 | AD | AD |
| AD21002 | AD | AD |
| AD21006 | AD | SQ |
| AD21011 | AD | AD |
| AD21012 | AD | AD |
| AD21013 | AD | AD |
| AD21014 | AD | AD |
| AD22003 | AD | AD |
| AD22005 | AD | SQ |
| AD22009 | AD | SQ |
| AD22010 | AD | AD |
| AD22037 | AD | AD |
| AD22048 | AD | AD |
| AD22051 | AD | AD |
| AD23005 | AD | AD |
| AD99015 | AD | AD |
| AD99034 | AD | AD |
| AD99035 | AD | AD |
| AD99043 | AD | AD |
| SM21015 | SM | SM |
| SM22060 | SM | SM |
| SQ22002 | SQ | SQ |
| SQ22004 | SQ | SQ |
| SQ22016 | SQ | SQ |
| SQ99011 | SQ | SQ |
| SQ99014 | SQ | SQ |

Definition of abbreviations:
AD = adenocarcinoma;
SM = small cell carcinoma;
SQ = Squamous cell carcinoma.

TABLE 4
SURVIVAL CLASSIFIERS

| Rank | Accession No. | Gene | Molecular Function |
|---|---|---|---|
| High risk | 1. 37724_at | MYC | Regulation of gene transcription |
| | 2. 1495_at | TGFB1 | Growth factor binding |
| | 3. 33439_at | SNF1LK | Protein tyrosine kinase |
| | 4. 35977_at | DKK1 | Signal transduction |
| | 5. 32065_at | CREM | Signal transduction |
| | 6. 33127_at | LOXL2 | Scavenger receptor activity |
| | 7. 39277_at | OSMR | DNA binding |
| | 8. 41049_at | IRS1 | Signal transduction |
| | 9. 34795_at | PLOD2 | Protein modification |
| | 10. 38422_s_at | FHL2 | Oncognesis |
| | 11. 35291_at | BAG2 | Chaperone activity |
| | 12. 36497_at | C14orf78 | |
| | 13. 37312_at | TRIP-Br2 | |
| | 14. 40074_at | MTHFD2 | Oxidoreductase activity |
| | 15. 32066_g_at | CREM | Signal transduction |
| | 16. 32186_at | SLC7AS | Amino acid transport |
| | 17. 34563_at | KIF14 | ATP binding |
| | 18. 37474_at | OIPS | Protein binding |
| | 19. 34777_at | ADM | Hormone activity |
| | 20. 31863_at | KIAA0179 | |
| | 21. 36873_at | VLDLR | Signal transduction |
| | 22. 547_s_at | NR4A2 | Transcription factor activity |
| | 23. 1973_s_at | MYC | Regulation of gene transcription |
| | 24. 41419_at | CED-6 | Signal transducer activity |
| | 25. 32067_at | CREM | Signal transduction |
| | 26. 41449_at | SGCE | Cell-matrix adhesion |
| | 27. 1945_at | CCNB1 | G₂/M transition of mitotic cell cycle |
| | 28. 37623_at | NR4A2 | Transcription factor activity |
| | 29. 34721_at | FKBP5 | FK506 binding |
| | 30. 33534_at | ESM1 | Insulin-like growth factor binding |
| Low risk | 1. 35207_at | SCNN1A | Ion channel activity |
| | 2. 39514_at | GADD45G | DNA repair |
| | 3. 37405_at | SELENBP1 | Selenium binding |
| | 4. 33754_at | TTF-1 | Transcription factor activity |
| | 5. 1664_at | HG3543-HT3739 | |
| | 6. 38095_i_at | HLA-DPB1 | Class II major histocompatibility complex |
| | 7. 38754_at | P8 | Induction of apoptosis |
| | 8. 33052_at | PLA2G10 | Phospholipase A_{z} activity |
| | 9. 39698_at | HOP | Transcription factor activity |
| | 10. 38028_at | DATI | |
| | 11. 41779_at | RGS16 | Signal transduction |
| | 12. 37021_at | CTSH | Cathepsin H activity |

### 7. EXAMPLE

### Class Prediction of Lung Nodule Gene Expression

### Profiles

Gene expression profiling is a powerful tool which may improve methods for risk stratification and treatment optimization in patients with lung cancer. We hypothesized that cellular material obtained at time of CT-guided biopsies of lung nodules could be used to generate clinically useful gene expression profiles.

Methods: Subjects were 18 patients undergoing CT-guided biopsy of undiagnosed pulmonary nodules. After biopsy of a lung nodule was performed and specimens were obtained for pathology, residual cells were placed into buffer for RNA extraction. Specimens were processed using the modified Eberwine protocol for analysis on the Affymetrix U95Av2 array, which contains probes for approximately 12,000 genes.

Results: To validate the small specimen amplification protocol, we compared the gene expression profiles generated by the modified Eberwine protocol using 100 nanograms of RNA with profiles obtained by standard amplification using 4 micrograms of RNA from the same tumor and found a correlation (r) of 0.82. We then generated gene expression profiles from 18 CT-guided biopsy specimens of lung nodules, which included 16 nonsmall cell cancers (NSCLC) and 2 nonmalignant lung samples. Class Prediction using K-nearest neighbor method in Gene Spring 5.0 was performed. We used 300 predictor genes and 3 nearest neighbors to predict histology. The training set consisted of 45 specimens (32 NSCLC, 7 nonmalignant lung and 6 mesotheliomas). Class Prediction analysis of the test set of CT-guided biopsy specimens accurately predicted the histology in 14 of 18 specimens. Specimens with incorrect classification included 2 NSCLC predicted to be nonmalignant lung, 1 NSCLC predicted to be a mesothelioma, and 1 nonmalignant lung predicted to be NSCLC.

Conclusions: Our data demonstrate that gene profiles of residual tissue from lung nodule biopsies accurately predict pathologic diagnosis. We plan to expand these studies with the goal of identifying marker genes predictive of treatment response and clinical outcome in patients with lung cancer.

### 8. EXAMPLE

### Extension of Survival Indicators to Other Cancers

To determine if the 42 Survival Classifiers were similarly associated with cancer outcome in other datasets, we examined a publicly available online database, Oncomine (Rhodes D R, Nature Genetics 2005; 37 Suppl:S31-7.) (www.oncomine.org). This database incorporates 132 independent datasets, totaling more than 10,000 microarray experiments, which span 24 cancer types. We examined differential activity for each gene, using a P value threshold of 0.001, focusing on phenotypes of survival and progression to metastasis. This analysis confirmed findings for the following 17 genes (Table 5). Column 1 indicates genes with expression associated with high risk of cancer death and column 2 indicates genes associated with low risk of cancer death. A summary of the Oncomine Analysis Results is depicted in Table 6.

TABLE 5

| High risk | Low risk |
|---|---|
| MYC | SCNN1A |
| TGFB1 | HLA-DPB1 |
| LOXL2 | DAT1 (LMO3) |
| IRS1 | CTSH |
| PLOD2 | |
| FHL2 | |
| TRIP-BR2 | |
| MTHFD2 | |
| SLC7A5 | |
| KIF14 | |
| ADM | |
| CCNB1 | |
| ESM1 | |

TABLE 6
Survival Classifiers - Oncomine Analysis Results Summary

| | | | |
|---|---|---|---|
| Gene | Phenotype | Tissue | Citation |

High Risk for Cancer Death

| | | | |
|---|---|---|---|
| 1 MYC | metastasis | prostrate | LaPointe, PNAS 2004 (49) |
| | metastasis | lung | Bhattarchee, PNAS 2001 (50) |
| | relapse | breast | Wang, Lancet 2005 (51) |
| 2 TGFB1 | metastasis | lung | Bhattarchee, PNAS 2001 (50) |
| | metastasis | lymphoma | Rosenwald, Cancer Cell 2003 (52) |
| 3 LOXL2 | metastasis | lung | Bhattarchee, PNAS 2001 (50) |
| | metastasis | renal | Boer, Genome Research 2001 (53) |
| 4 IRS1 | metastasis | lung | Bhattarchee, PNAS 2001 (50) |
| | metastasis | prostate | LaPointe, PNAS 2004 (49) |
| | metastasis | prostate | Yu, J. Clin Onc 2004 (54) |
| 5 PLOD2 | metastasis | prostate | Yu, J. Clin Onc 2004 (54) |
| 6 FHL2 | metastasis | prostate | LaPointe, PNAS 2004 (49) |
| | metastasis | prostate | Yu, J. Clin Onc 2004 (54) |
| | Gleason score | prostate | Singh, Cancer Cell 2002 (55) |
| 7 TRIP-2BR | | | |
| 8 MTHFD2 | metastasis | prostate | LaPointe, PNAS 2004 (49) |
| | metastasis | prostate | Yu, J. Clin Onc 2004 (54) |
| 9 SLC7A5 | metastasis | breast | vandeVijver, NEJM 2002 (56) |
| | metastasis | prostate | Yu, J. Clin Onc. 2004 (54) |
| | metastasis | melanoma | Haqq, PNAS 2005 (57) |
| 10 KIF14 | metastasis | prostate | Yu, J. Clin Onc 2004 (54) |
| 11 ADM | metastasis | prostate | Yu, J. Clin Onc 2004 (54) |
| | metastasis | prostate | Dhanasekaran, Nature 2001 |
| | | | (58) |
| | metastasis | breast | vandeVijver, NEJM 2002 (56) |
| 12 CCNB1 | metastasis | prostate | Yu, J. Clin Onc 2004 (54) |
| | metastasis | prostate | LaTulippe, Can Res 2002 (59) |
| | metastasis | prostate | Dhanasekaran, Nature 2001 |
| | | | (58) |
| | relapse | breast | vandeVijver, NEJM 2002 (56) |
| | metastasis | breast | vandeVijver, NEJM 2002 (56) |
| 13 ESM1 | death | brain | Freije, Can Res 2004 (60) |

Low Risk for Cancer Death

| | | | |
|---|---|---|---|
| 14 SCNN1A | metastasis | prostate | LaPointe, PNAS 2004 (49) |
| 15 HLA-DPB1 | metastasis | lung, ovarian, | Ramaswamy, PNAS 2001 |
| | | prostate | (61) |
| | metastasis | prostate | Yu, J Clin Onc 2004 (54) |
| | metastasis | prostate | Dhanasekaran, Nature 2001 |
| | | | (58) |

High Risk for Cancer Death

| | | | |
|---|---|---|---|
| 16 DAT1 (LMO3) | metastasis | lung, prostate | Ramaswamy, PNAS 2001 |
| | | | (61) |
| 17 CTSH | metastasis | prostate | Dhanasekaran, Nature 2001 (58) |

### 9. REFERENCES

1. Jemal A, Tiwari R C, Murray T, Ghafoor A, Samuels A, Ward E, Feuer E J, and Thun M J. Cancer Statistics, 2004. CA Cancer J Clin 2004; 54:8-29.
2. Waters J S, and O'Brien M E. The case for the introduction of new chemotherapy agents in the treatment of advanced non small cell lung cancer in the wake of the findings of The National Institute of Clinical Excellence (NICE). Br J Cancer 2002; 87:481-490.
3. Spiro S G, and Porter J C. Lung Cancer—Where Are We Today?: Current Advances in Staging and Nonsurgical Treatment. Am. J. Respir. Crit. Care Med. 2002; 166:1166-1196.
4. Powell C A, Spira A, Derti A, et al. Gene Expression in Lung Adenocarcinomas of Smokers and Nonsmokers. Am. J. Respir. Cell Mol. Biol. 2003; 29:157-162.
5. Sugita M, Geraci M, Gao B, et al. Combined use of oligonucleotide and tissue microarrays identifies cancer/testis antigens as biomarkers in lung carcinoma. Cancer Res 2002; 62:3971-3979.
6. Borczuk A C, Gorenstein L, Walter K L, Assaad A A, Wang L, and Powell C A. Non-small-cell lung cancer molecular signatures recapitulate lung developmental pathways. Am J Pathol 2003; 163:1949-1960.
7. Bhattacharjee A, Richards W G, Staunton J, et al. Classification of human lung carcinomas by mRNA expression profiling reveals distinct adenocarcinoma subclasses. Proc Natl Acad Sci USA 2001; 98:13790-13795.
8. Beer D G, Kardia S L, Huang C C, et al. Gene-expression profiles predict survival of patients with lung adenocarcinoma. Nat Med 2002; 8:816-824.
9. Datta D, and Lahiri B. Preoperative evaluation of patients undergoing lung resection surgery. Chest 2003; 123:2096-2103.
10. British Thoracic Society guidelines on diagnostic flexible bronchoscopy. Thorax 2001; 56 Suppl 1:i1-21.
11. Ernst A, Silvestri G A, and Johnstone D. Interventional pulmonary procedures: Guidelines from the American College of Chest Physicians. Chest 2003; 123:1693-1717.
12. Geraghty P R, Kee S T, McFarlane G, Razavi M K, Sze D Y, and Dake M D. CT-guided transthoracic needle aspiration biopsy of pulmonary nodules: needle size and pneumothorax rate. Radiology 2003; 229:475-481.
13. Kazerooni E A, Lim F T, Mikhail A, and Martinez F J. Risk of pneumothorax in CT-guided transthoracic needle aspiration biopsy of the lung. Radiology 1996; 198:371-375.
14. Walter K L, Borczuk A C, Wang L, Assaad A M, Austin J H M, Pearson G D N, Shiau M C, and Powell C A. Class Prediction of Lung Nodule Gene Expression Profiles. Chest 2004; 125:In Press.
15. Kacharmina J E, Crino P B, and Eberwine J. Preparation of cDNA from single cells and subcellular regions. Methods Enzymol 1999; 303:3-18.
16. Irizarry R A, Bolstad B M, Collin F, Cope L M, Hobbs B, and Speed T P. Summaries of Affymetrix GeneChip probe level data. Nucleic Acids Res 2003; 31:e15.
17. Simon R, Radmacher R, and Bittner M. 2003. BRB Tools. 3.0 ed. National Cancer Institute.
18. Simon R, Radmacher M D, Dobbin K, and McShane LM. Pitfalls in the use of DNA microarray data for diagnostic and prognostic classification. J. Natl. Cancer Inst. 2003; 95:14-18.
19. Lyons-Weiler J, Patel S, and Bhattacharya S. A classification-based machine learning approach for the analysis of genome-wide expression data. Genome Res 2003; 13:503-512.
20. Travis W D, Colby T V, Corrin B, Shimosato Y, and Brambilla E. World Health Organization International Histological Classification of Tumours. Histological Typing of Lung and Pleural Tumors., 3rd ed. New York: Springer-Verlag; 1999.
21. Mantel N. Evaluation of survival data and two new rank order statistics arising in its consideration. Cancer Chemother Rep 1966; 50:163-170.
22. Meier P, and Kaplan E. Nonparametric estimation from incomplete observations. J Am Stat Assoc 1958; 158:457-481.
23. Sotiriou C, Powles T J, Dowsett M, Jazaeri A A, Feldman A L, Assersohn L, Gadisetti C, Libutti S K, and Liu E T. Gene expression profiles derived from fine needle aspiration correlate with response to systemic chemotherapy in breast cancer. Breast Cancer Res 2002; 4:R3.
24. Luzzi V, Mahadevappa M, Raja R, Warrington J A, and Watson M A. Accurate and reproducible gene expression profiles from laser capture microdissection, transcript amplification, and high density oligonucleotide microarray analysis. J Mol Diagn 2003; 5:9-14.
25. Symmans W F, Ayers M, Clark E A, et al. Total RNA yield and microarray gene expression profiles from fine-needle aspiration biopsy and core-needle biopsy samples of breast carcinoma. Cancer 2003; 97:2960-2971.
26. Wright G W, and Simon R M. A random variance model for detection of differential gene expression in small microarray experiments. Bioinformatics 2003; 19:2448-2455.
27. Pedersen N, Mortensen S, Sorensen S B, Pedersen M W, Rieneck K, Bovin L F, and Poulsen H S. Transcriptional gene expression profiling of small cell lung cancer cells. Cancer Res 2003; 63:1943-1953.
28. Duda R O, Hart P E, and Stork D G. Pattern Classification, 2nd ed. New York: Wiley; 2001.
29. Saksela K, Bergh J, Lehto V P, Nilsson K, and Alitalo K. Amplification of the c-myc oncogene in a subpopulation of human small cell lung cancer. Cancer Res 1985; 45:1823-1827.
30. Schlotter C M, Vogt U, Bosse U, Mersch B, and Wassmann K. C-myc, not HER-2/neu, can predict recurrence and mortality of patients with node-negative breast cancer. Breast Cancer Res 2003; 5:R30-36.
31. Nagy B, Lundan T, Larramendy M L, et al. Abnormal expression of apoptosis-related genes in haematological malignancies: overexpression of MYC is poor prognostic sign in mantle cell lymphoma. Br J Haematol 2003; 120:434-441.
32. Takeno S, Noguchi T, Kikuchi R, Uchida Y, Yokoyama S, and Muller W. Prognostic value of cyclin B 1 in patients with esophageal squamous cell carcinoma. Cancer 2002; 94:2874-2881.
33. Soria J C, Jang S J, Khuri F R, Hassan K, Liu D, Hong W K, and Mao L. Overexpression of cyclin B1 in early-stage non-small cell lung cancer and its clinical implication. Cancer Res 2000; 60:4000-4004.
34. Wei Y, Renard C-A, Labalette C, Wu Y, Levy L, Neuveut C, Prieur X, Flajolet M, Prigent S, and Buendia M-A. Identification of the LIM Protein FHL2 as a Coactivator of beta-Catenin. J. Biol. Chem. 2003; 278:5188-5194.
35. Keum J S, Kong G, Yang S C, Shin D H, Park S S, Lee J H, and Lee J D. Cyclin D1 overexpression is an indicator of poor prognosis in resectable non-small cell lung cancer. Br J Cancer 1999; 81:127-132.
36. Rimsza L M, Roberts R A, Miller T P, et al. Loss of MHC Class II Gene and Protein Expression in Diffuse Large B Cell Lymphoma is Related to Decreased Tumor Immunosurveillance and Poor Patient Survival Irrespective of other Prognostic Factors: A Follow-up Study from the Leukemia and Lymphoma Molecular Profiling Project. Blood 2004:2003-2007-2365.
37. Jemal A, Murray T, Samuels A, Ghafoor A, Ward E, and Thun M J. Cancer statistics, 2003. CA Cancer J Clin 2003; 53:5-26.
38. Pisters K M, Ginsberg R J, Giroux D J, Putnam J B, Jr., Kris M G, Johnson D H, Roberts J R, Mault J, Crowley J J, and Bunn P A, Jr. Induction chemotherapy before surgery for early-stage lung cancer: A novel approach. Bimodality Lung Oncology Team. J Thorac Cardiovasc Surg 2000; 119:429-439.
39. Le Chevalier T. Results of the Randomized International Adjuvant Lung Cancer Trial (IALT): Cisplatin-based chemotherapy vs no CT in 1867 patients with resected non-small cell lung cancer. J Clin Oncol 2003; 21:238.
40. Chang J C, Wooten E C, Tsimelzon A, et al. Gene expression profiling for the prediction of therapeutic response to docetaxel in patients with breast cancer. Lancet 2003; 362:362-369.
41. Staunton J E, Slonim D K, Coller H A, et al. Chemosensitivity prediction by transcriptional profiling. Proc Natl Acad Sci USA 2001; 98:10787-10792.
42. Scherf U, Ross D T, Waltham M, et al. A gene expression database for the molecular pharmacology of cancer. Nat Genet 2000; 24:236-244.
43. Rosenwald A, Wright G, Chan W C, et al. The use of molecular profiling to predict survival after chemotherapy for diffuse large-B-cell lymphoma. N Engl J Med 2002; 346:1937-1947.
44. Shipp M A, Ross K N, Tamayo P, et al. Diffuse large B-cell lymphoma outcome prediction by gene-expression profiling and supervised machine learning. Nat Med 2002; 8:68-74.
45. Lim E H, Aggarwal A, Agasthian T, et al. Feasibility of using low-volume tissue samples for gene expression profiling of advanced non-small cell lung cancers. Clin Cancer Res 2003; 9:5980-5987.
46. Swensen S J, Jett J R, Sloan J A, et al. Screening for lung cancer with low-dose spiral computed tomography. Am J Respir Crit Care Med 2002; 165:508-513.
47. Borczuk A C, Shah L, Pearson G D N, Walter K L, Wang L, Austin J H M, Friedman R A and Powell C A. Molecular signatures in biopsy specimens of lung cancer. Am. J. Respiratory Critical Care Med. 2004, 170: 167-174.
48. Ding C and Cantor C, A high-throughput gene expression analysis technique using competitive PCR and matrix-assisted laser desorption ionization time-of-flight MS. Proc. Natl. Acad. Sci. U.S.A. 2003, 100:3059-3064.
49. LaPoint et al., 2004, Gene expression profiling identifies clinically relevant subtypes of prostate cancer. Proc Natl Acad Sci U S A. 101(3):811-6. Epub 2004 Jan. 7.
50. Bhattacharjee et al., 2001, Classification of human lung carcinomas by mRNA expression profiling reveals distinct adenocarcinoma subclasses. Proc Natl Acad Sci U S A. 98(24):13790-5. Epub 2001 Nov. 13.
51. Wang et al., 2005 Gene-expression profiles to predict distant metastasis of lymph-node-negative primary breast cancer. Lancet. 365(9460):671-9.
52. Rosenwald et al., 2003 The proliferation gene expression signature is a quantitative integrator of oncogenic events that predicts survival in mantle cell lymphoma. Cancer Cell. (2): 185-97.
53. Boer et al., 2001 Identification and classification of differentially expressed genes in renal cell carcinoma by expression profiling on a global human 31,500-element cDNA array. Genome Res. 11(11):1861-70.
54. Yu et al., 2004 Gene expression alterations in prostate cancer predicting tumor aggression and preceding development of malignancy. J Clin Oncol. 22(14):2790-9.
55. Singh et al., 2002 Cancer Cell. 1(2):203-9. Gene expression correlates of clinical prostate cancer behavior.
56. Van de Vijver 2002 A gene-expression signature as a predictor of survival in breast cancer. N Engl J Med. 347(25):1999-2009.
57. Haqq et al., 2005 The gene expression signatures of melanoma progression. Proc Natl Acad Sci USA. 102(17):6092-7. Epub 2005 Apr. 15.
58. Dhanashekaran et al., 2001 Delineation of prognostic biomarkers in prostate cancer. Nature. 412(6849):822-6.
59. LaTulippe et al., 2002 Comprehensive gene expression analysis of prostate cancer reveals distinct transcriptional programs associated with metastatic disease. Cancer Res. 62(15):4499-506.
60. Freije et al., 2004 Gene expression profiling of gliomas strongly predicts survival. Cancer Res. 64(18):6503-10.
61. Ramaswamy et al., 2001 Multiclass cancer diagnosis using tumor gene expression signatures. Proc Natl Acad Sci U S A 98(26): 15149-54. Epub 2001 Dec. 11. Related Articles, Links
62. Rhodes et al., 2005 Integrative analysis of the cancer transcriptome. Nat Genet. 37 Suppl:S31-7. Review.
63. Rhodes et al., Mining for regulatory programs in the cancer transcriptome. Nat Genet. 37(6):579-83.

Various publications are cited above, the contents of which are hereby incorporated by reference in their entireties.

We claim:

1. A method for evaluating the histology of a sample comprising lung cells, comprising measuring, in the sample, the expression of a plurality of genes selected from the group consisting of RPS6KA2, BAIAP2, IL1R1, ASL, PRSS8, DAT1, HPN, PHF15, FLJ12443, HLA-DPB1, HOP, LGALS3BP, RUNX1, RBPMS, C11 orf9, HFL1, CEACAM1, RABL4, CAPN2, CLDN4, PON2, MICAL2, GPR116, FLJI2443, NpC2, WSB1, CPD, CASP8, STEAP, FOS, TRIM38 and ALOX15B, wherein a relative increase in the expression of such genes has a positive correlation with the presence of lung adenocarcinoma cells.

2. A method for evaluating the histology of a sample comprising lung cells, comprising measuring, in the sample, the expression of a plurality of genes selected from the group consisting of DKFZp564N1662, SH3GL3, GNAZ, MEIS2, ELOVL2, AF038185, RELN, C11 orf8, AF1Q, KIAA0535, BCL11A, NY-ESO-1, SEPHS1, CDKNIC, BAT8, RIMS2, HEC, FLJ36166, APBA2, TCF3, EYA2, RBP1, L-myc, CDKN2A, SFPQ, KIFC1, ZNF339, CRABP1, RANBP1, STMN1, NCAD, FLJ12377, LMNB1, MGC51028, CENPF, MCM2, INSM1, VRK1, UCHL1, P311, BLM, BCL11A, BCL2, INA, and KIAA0186, wherein a relative increase in the expression of such genes has a positive correlation with the presence of small cell lung carcinoma cells.

3. A method for evaluating the histology of a sample comprising lung cells, comprising measuring, in the sample, the expression of a plurality of genes selected from the group consisting of C4.4A, SAP-3, FST, TRIM29, PTPRC, wherein a relative increase in the expression of such genes has a positive correlation with the presence of squamous cell lung carcinoma cells.

4. A method for evaluating the prognosis of a patient suffering from lung cancer, comprising measuring, in a tumor sample from the patient, the expression of a plurality of genes selected from the group consisting of MYC, TGFB1, SNF1LK, DKK1, LOXL2, OSMR, IRS1, PLOD2, FHL2, BAG2, C14orf78, TRIP-Br2, MTHFD2, SLC7A5, KIF14, OIP5, ADM, KIAA0179, VLDLR, NR4A2, CED-6, CREM, SGCE, CCNB1, NR4A2, FKBP5, and ESM1, wherein a relative increase in the expression of such genes has a positive correlation with a higher risk of shortened survival.

5. The method of claim 4, comprising measuring the expression of genes selected from the group consisting of CCNB1, FHL2, LOXL2, IRS1, PLOD2, MTHFD2, TGFB1 and TRIPBR2.

6. A method for evaluating the prognosis of a patient suffering from lung cancer, comprising measuring, in a tumor sample from the patient, the expression of a plurality of genes selected from the group consisting of SCNN1A, GADD45G, SELENBP1, TTF-1, HG3543-HT3739, HLA-DPB1, P8, PLA2G10, HOP, DATI, RGS16, and CTSH, wherein a relative increase in the expression of such genes has a positive correlation with a lower risk of shortened survival.

7. The method of claim 6, comprising measuring the expression of HLA-DPB1.

8. A kit for evaluating a lung tumor sample comprising a plurality of oligonucleotides that specifically bind to a plurality of genes selected from the group consisting of RPS6KA2, BAIAP2, IL1R1, ASL, PRSS8, DAT1, HPN, PHF15, FLJ12443, HLA-DPB1, HOP, LGALS3BP, RUNX1, RBPMS, C11 orf9, HFL1, CEACAM1, RABL4, CAPN2, CLDN4, PON2, MUC1, MICAL2, GPR116, FLJ12443, NpC2, WSB1, CPD, CASP8, STEAP, FOS, TRIM38, ALOX15B, DKFZp564N1662, SH3GL3, GNAZ, MEIS2, ELOVL2, AF038185, RELN, C11 orf8, AF1Q, KIAA0535, BCL11A, NY-ESO-1, SEPHS1, CDKNIC, BAT8, RIMS2, HEC, FLJ36166, APBA2, TCF3, EYA2, RBP1, L-myc, CDKN2A, SFPQ, KIFC1, ZNF339, CRABP1, RANBP1, STMN1, NCAD, FLJ12377, LMNB1, MGC51028, CENPF, MCM2, INSM1, VRK1, UCHL1, P311, BLM, BCL11A, BCL2, INA, KIAA0186, C4.4A, SAP-3, FST, TRIM29, PTPRC, MYC, TGFB1, SNF1LK, DKK1, LOXL2, OSMR, IRS1, PLOD2, FHL2, BAG2, C14orf78, TRIP-Br2, MTHFD2, SLC7A5, KIF14, OIP5, ADM, KIAA0179, VLDLR, NR4A2, CED-6, CREM, SGCE, CCNB1, NR4A2, FKBP5, ESM1, SCNN1A, GADD45G, SELENBP1, TTF-1, HG3543-HT3739, HLA-DPB1, P8, PLA2G10, HOP, DAT1, RGS16, and CTSH.

9. The kit of claim 8, where at least one of the oligonucleotides is detectably labeled.

10. The kit of claim 8, wherein at least two of the oligonucleotides constitute a primer pair which may be used in a polymerase chain reaction.

11. A kit for evaluating a lung tumor sample comprising a matrix to which is bound a nucleic acid corresponding to each of a plurality of genes selected from the group consisting of RPS6KA2, BAIAP2, IL1R1, ASL, PRSS8, DAT1, HPN, PHF15, FLJ12443, HLA-DPB1, HOP, LGALS3BP, RUNX1, RBPMS, C11 orf9, HFL1, CEACAM1, RABL4, CAPN2, CLDN4, PON2, MUC1, MICAL2, GPR116, FLJI2443, NpC2, WSB1, CPD, CASP8, STEAP, FOS, TRIM38, ALOX15B, DKFZp564N1662, SH3GL3, GNAZ, MEIS2, ELOVL2, AF038185, RELN, C11 orf8, AF1Q, KIAA0535, BCL11A, NY-ESO-1, SEPHS1, CDKNIC, BAT8, RIMS2, HEC, FLJ36166, APBA2, TCF3, EYA2, RBP1, L-myc, CDKN2A, SFPQ, KIFC1, ZNF339, CRABP1, RANBP1, STMN1, NCAD, FLJ12377, LMNB1, MGC51028, CENPF, MCM2, INSM1, VRK1, UCHL1, P311, BLM, BCL11A, BCL2, INA, KIAA0186, C4.4A, SAP-3, FST, TRIM29, PTPRC, MYC, TGFB1, SNF1LK, DKK1, LOXL2, OSMR, IRS1, PLOD2, FHL2, BAG2, C14orf78, TRIP-Br2, MTHFD2, SLC7A5, KIF14, OIP5, ADM, KIAA0179, VLDLR, NR4A2, CED-6, CREM, SGCE, CCNB1, NR4A2, FKBP5, ESM1, SCNN1A, GADD45G, SELENBP1, TTF-1, HG3543-HT3739, HLA-DPB1, P8, PLA2G10, HOP, DAT1, RGS16, and CTSH, wherein the number of gene species represented by said plurality of genes constitutes a majority of the total number of gene species bound to the matrix.

12. A kit for practicing the method of claim 1 comprising a plurality of oligonucleotides that specifically bind to a plurality of genes selected from the group consisting of RPS6KA2, BAIAP2, IL1R1, ASL, PRSS8, DAT1, HPN, PHF15, FLJ12443, HLA-DPB1, HOP, LGALS3BP, RUNX1, RBPMS, C11 orf9, HFL1, CEACAM1, RABL4, CAPN2, CLDN4, PON2, MICAL2, GPR116, FLJI2443, NpC2, WSB1, CPD, CASP8, STEAP, FOS, TRIM38 and ALOX15B, wherein said plurality of genes are identified as lung adenocarcinoma associated genes.

13. The kit of claim 12, where at least one of the oligonucleotides is detectably labeled.

14. The kit of claim 12, wherein at least two of the oligonucleotides constitute a primer pair which may be used in a polymerase chain reaction.

15. A kit for practicing the method of claim 1 comprising a matrix to which is bound a nucleic acid corresponding to each of a plurality of genes selected from the group consisting of RPS6KA2, BAIAP2, IL1R1, ASL, PRSS8, DAT1, HPN, PHF15, FLJ12443, HLA-DPB1, HOP, LGALS3BP, RUNX1, RBPMS, C11 orf9, HFL1, CEACAM1, RABL4, CAPN2, CLDN4, PON2, MICAL2, GPR116, FLJ12443, NpC2, WSB1, CPD, CASP8, STEAP, FOS, TRIM38 and ALOX15B, wherein said plurality of genes are identified as lung adenocarcinoma associated genes.

16. A kit for practicing the method of claim 2 comprising a plurality of oligonucleotides that specifically bind to a plurality of genes selected from the group consisting of DKFZp564N1662, SH3GL3, GNAZ, MEIS2, ELOVL2, AF038185, RELN, C11 orf8, AF1Q, KIAA0535, BCL11A, NY-ESO-1, SEPHS1, CDKNIC, BAT8, RIMS2, HEC, FLJ36166, APBA2, TCF3, EYA2, RBP1, L-myc, CDKN2A, SFPQ, KIFC1, ZNF339, CRABP1, RANBP1, STMN1, NCAD, FLJ12377, LMNB1, MGC51028, CENPF, MCM2, INSM1, VRK1, UCHL1, P311, BLM, BCL11A, BCL2, INA, and KIAA0186, wherein said plurality of genes are identified as small cell lung carcinoma associated genes.

17. The kit of claim 16, where at least one of the oligonucleotides is detectably labeled.

18. The kit of claim 16, wherein at least two of the oligonucleotides constitute a primer pair which may be used in a polymerase chain reaction.

19. A kit for practicing the method of claim 2 comprising a matrix to which is bound a nucleic acid corresponding to each of a plurality of genes selected from the group consisting of DKFZp564N1662, SH3GL3, GNAZ, MEIS2, ELOVL2, AF038185, RELN, C11 orf8, AF1Q, KIAA0535, BCL11A, NY-ESO-1, SEPHS1, CDKNIC, BAT8, RIMS2, HEC, FLJ36166, APBA2, TCF3, EYA2, RBP1, L-myc, CDKN2A, SFPQ, KIFC1, ZNF339, CRABP1, RANBP1, STMN1, NCAD, FLJ12377, LMNB1, MGC51028, CENPF, MCM2, INSM1, VRK1, UCHL1, P311, BLM, BCL11A, BCL2, INA, and KIAA0186, wherein said plurality of genes are identified as small cell lungcarcinoma associated genes.

20. A kit for practicing the method of claim 3 comprising a plurality of oligonucleotides that specifically bind to a plurality of genes selected from the group consisting of C4.4A, SAP-3, FST, TRIM29, PTPRC, wherein said plurality of genes are identified as squamous cell lung carcinoma associated genes.

21. The kit of claim 20, where at least one of the oligonucleotides is detectably labeled.

22. The kit of claim 20, wherein at least two of the oligonucleotides constitute a primer pair which may be used in a polymerase chain reaction.

23. A kit for practicing the method of claim 3 comprising a matrix to which is bound a nucleic acid corresponding to each of a plurality of genes selected from the group consisting of C4.4A, SAP-3, FST, TRIM29, PTPRC, wherein said plurality of genes are identified as squamous cell lung carcinoma associated genes.

24. A kit for practicing the method of claim 4 comprising a plurality of oligonucleotides that specifically bind to a plurality of genes selected from the group consisting of MYC, TGFB1, SNF1LK, DKK1, LOXL2, OSMR, IRS1, PLOD2, FHL2, BAG2, C14orf78, TRIP-Br2, MTHFD2, SLC7A5, KIF14, OIP5, ADM, KIAA0179, VLDLR, NR4A2, CED-6, CREM, SGCE, CCNB1, NR4A2, FKBP5, and ESM1, wherein said plurality of genes are identified as shortened survival associated genes.

25. The kit of claim 24, where at least one of the oligonucleotides is detectably labeled.

26. The kit of claim 24, wherein at least two of the oligonucleotides constitute a primer pair which may be used in a polymerase chain reaction.

27. A kit for practicing the method of claim 4 comprising a matrix to which is bound a nucleic acid corresponding to each of a plurality of genes selected from the group consisting of MYC, TGFB1, SNF1LK, DKK1, LOXL2, OSMR, IRS1, PLOD2, FHL2, BAG2, C14orf78, TRIP-Br2, MTHFD2, SLC7A5, KIF14, OIP5, ADM, KIAA0179, VLDLR, NR4A2, CED-6, CREM, SGCE, CCNB1, NR4A2, FKBP5, and ESM1, wherein said plurality of genes are identified as shortened survival associated genes.

28. A kit for practicing the method of claim 6 comprising a plurality of oligonucleotides that specifically bind to a plurality of genes selected from the group consisting of SCNN1A, GADD45G, SELENBP1, TTF-1, HG3543-HT3739, HLA-DPB1, P8, PLA2G10, HOP, DAT1, RGS16, and CTSH, wherein said plurality of genes are identified as lower risk of shortened survival associated genes.

29. The kit of claim 28, where at least one of the oligonucleotides is detectably labeled.

30. The kit of claim 28, wherein at least two of the oligonucleotides constitute a primer pair which may be used in a polymerase chain reaction.

31. A kit for practicing the method of claim 6 comprising a matrix to which is bound a nucleic acid corresponding to each of a plurality of genes selected from the group consisting of SCNN1A, GADD45G, SELENBP1, TTF-1, HG3543-HT3739, HLA-DPB1, P8, PLA2G10, HOP, DAT1, RGS16, and CTSH, wherein said plurality of genes are identified as lower risk of shortened survival associated genes.

32. A method for evaluating the prognosis of a patient suffering from a cancer other than lung cancer, comprising measuring, in a tumor sample from the patient, the expression of a plurality of genes selected from the group consisting of MYC, TGFB1, LOXL2, IRS1, PLOD2, FHL2, TRIP-BR2, MTHFD2, SLC7A5, KIF14, ADM, CCNB1 and ESM1, wherein a relative increase in the expression of such genes has a positive correlation with a shorter survival relative to that of a patient having a tumor in which the expression of said genes is not increased.

33. A method for evaluating the prognosis of a patient suffering from a cancer which is not lung cancer, comprising measuring, in a tumor sample from the patient, the expression of a plurality of genes selected from the group consisting of SCNNIA, HLA-DPB1, DAT1 (LMO3) and CTSH, wherein a relative increase in the expression of such gene or genes has a positive correlation with a longer survival relative to that of a patient having a tumor in which the expression of said genes is not increased.

34. A kit for evaluating a tumor sample comprising a plurality of oligonucleotides that specifically bind to a plurality of genes selected from the group consisting of MYC, TGFB1, LOXL2, IRS1, PLOD2, FHL2, TRIP-BR2, MTHFD2, SLC7A5, KIF14, ADM, CCNB1 and ESM1, wherein said plurality of genes are identified as shorter survival associated genes.

35. The kit of claim 34, where at least one of the oligonucleotides is detectably labeled.

36. The kit of claim 34, wherein at least two of the oligonucleotides constitute a primer pair which may be used in a polymerase chain reaction.

37. A kit for evaluating a tumor sample comprising a matrix to which is bound a nucleic acid corresponding to each of a plurality of genes selected from the group consisting of MYC, TGFB1, LOXL2, IRS1, PLOD2, FHL2, TRIP-BR2, MTHFD2, SLC7A5, KIF14, ADM, CCNB1 and ESM1, wherein said plurality of genes are identified as shortened survival associated genes.

38. A kit for evaluating a tumor sample comprising a plurality of oligonucleotides that specifically bind to a plurality of genes selected from the group consisting of SCNNIA, HLA-DPB1, DAT1 (LMO3) and CTSH, wherein said plurality of genes are identified as longer survival associated genes.

39. The kit of claim 38, where at least one of the oligonucleotides is detectably labeled.

40. The kit of claim 38, wherein at least two of the oligonucleotides constitute a primer pair which may be used in a polymerase chain reaction.

41. A kit for evaluating a tumor sample comprising a matrix to which is bound a nucleic acid corresponding to each of a plurality of genes selected from the group consisting of SCNNIA, HLA-DPB1, DAT1 (LMO3) and CTSH, wherein said plurality of genes are identified as longer survival associated genes.
